(19) United States
(12) Patent Application Publication
(10) Pub. No.: US 2019/0201061 A1
(43) Pub. Date: Jul. 4, 2019

(54) EXPANDABLE ORTHOPEDIC IMPLANT

(71) Applicants: INDUSTRIAL TECHNOLOGY RESEARCH INSTITUTE, Hsinchu (TW); National Taiwan University Hospital, Taipei City (TW)

(72) Inventors: Pei-I TSAI, Hsinchu City (TW); Hsin-Hsin SHEN, Hsinchu County (TW); Kuo-Yi YANG, Hsinchu City (TW); Shih-Ping LIN, Kaohsiung City (TW); Jui-Sheng SUN, Taipei City (TW); Chih-Yu CHEN, Zhubei City (TW); Yu-Tsung CHIU, Taipei City (TW); Chun-Ti CHEN, Hsinchu City (TW); De-Yau LIN, Tainan City (TW); An-Li CHEN, Tainan City (TW)

(73) Assignees: INDUSTRIAL TECHNOLOGY RESEARCH INSTITUTE, Hsinchu (TW); National Taiwan University Hospital, Taipei City (TW)

(21) Appl. No.: 15/856,774

(22) Filed: Dec. 28, 2017

(51) Int. Cl. A61B 17/72; A61B 17/74

(52) U.S. Cl. CPC A61B 17/7266; A61B 17/8805; A61B 17/746

(57) ABSTRACT

An expandable orthopedic implant includes a casing pipe, a shaft, a first expandable element and a first link lever. The casing pipe has a first opening, a second opening and an axis passing through the first and second opening. The casing pipe sheathes one end of the shaft, and the other end of the shaft has a top portion disposed out of the first opening. The first expandable element has a first terminal pivoted on the casing pipe and a second terminal. The first link lever has a proximal end pivoted to the top portion and a distal end pivoted to the second terminal. When the top portion is driven moving away from the first opening along an extending direction of the axis, the first link-lever can be pushed by the shaft to enact the second terminal moving away from the axis along a direction perpendicular to the axis.

### TECHNICAL FIELD

The disclosure relates in general to an implantable medical device; and more particularly to an expandable orthopedic implant device.

### BACKGROUND

From the clinical observation it can be found that, an expandable orthopedic implant device applied in an orthopedic surgery for stabilizing or fixing the fractured bone may be failure due to superior cutout which is caused by loss of bone density and osteoporosis of the patients. To take an orthopedic internal fixation device used for treating intertrochanteric fractures, such as a dynamic hip screw (DHS), a gamma nail or a sliding plate screw, as an example, a DHS that is implanted into a patient's femur may be failure, destruction and loosening and lead collapse of the femoral head, when the bone density of the patient is too low to sustain the impact of the orthopedic internal fixation device.

Therefore, there is a need of providing an expandable orthopedic implant device to obviate the drawbacks encountered from the prior art.

### SUMMARY

According to one embodiment of the present disclosure, an expandable orthopedic implant device is provided, wherein the expandable orthopedic implant device includes a casing pipe, a shaft, a first expandable element and a first link-lever. The casing pipe has a first opening, a second opening and an axis passing through the first and second opening. The casing pipe sheathes one end of the shaft, and the other end of the shaft has a head portion disposed out of the first opening. The first expandable element has a first terminal pivoted on the casing pipe and a second terminal. The first link-lever has a proximal end pivoted to the head portion and a distal end pivoted to the second terminal. When the head portion is driven moving away from the first opening along an extending direction of the axis, the first link-lever can be pushed by the shaft to enact the second terminal moving away from the axis along a direction perpendicular to the axis.

According to another embodiment of the present disclosure, an expandable orthopedic implant device including a shaft, a casing pipe sheathing one end of the shaft, an expandable element and a link-lever is provided, wherein a distal end of the link-lever is pivoted to a second terminal of the expandable element, and a proximal end of the link-lever and a first terminal of the expandable element opposite to the second terminal are respectively pivoted to the shaft and the casing pipe. When the second terminal of the expandable element is driven by the link-lever enacted by the shaft moving away from the axis along a direction perpendicular to the axis, an anchor structure can be formed by the expandable element and the link-lever to increase the contact area and friction between the expandable orthopedic implant device and the peripheral tissues of the implanted bone, so as to prevent the expandable orthopedic implant device from rotating or bonding loosening, whereby the mechanical stability of the expandable orthopedic implant device implanted in bones can be improved, and the risk of superior cutout can be significantly reduced.

### BRIEF DESCRIPTION OF THE DRAWINGS

The disclosure can be more fully understood by reading the subsequent detailed description and examples with references made to the accompanying drawings, wherein:

FIG. 1A is a cross-sectional view illustrating an unexpanded expandable orthopedic implant device according to one embodiment of the present disclosure;

FIG. 1B is a cross-sectional view illustrating partial enlarged detail of the unexpanded expandable orthopedic implant device as depicted in FIG. 1A;

FIG. 1C is a prospective view illustrating the expanded form of the expandable orthopedic implant device as depicted in FIG. 1A;

FIG. 2A is a cross-sectional view illustrating an unexpanded expandable orthopedic implant device according to another embodiment of the present disclosure;

FIG. 2B is a prospective view illustrating the expanded form of the expandable orthopedic implant device as depicted in FIG. 2A;

FIG. 3A is a cross-sectional view illustrating an unexpanded expandable orthopedic implant device according to yet another embodiment of the present disclosure;

FIG. 3B is a prospective view illustrating the expanded form of the expandable orthopedic implant device as depicted in FIG. 3A;

FIG. 4A is a cross-sectional view illustrating an unexpanded expandable orthopedic implant device according to a fourth embodiment of the present disclosure;

FIG. 4B is a cross-sectional view illustrating partial enlarged detail of the unexpanded expandable orthopedic implant device as depicted in FIG. 4A;

FIG. 4C is a prospective view illustrating the expanded form of the expandable orthopedic implant device as depicted in FIG. 4A;

FIG. 5 is a prospective view illustrating the expanded form of an expandable orthopedic implant device according to a fifth embodiment of the present disclosure;

FIG. 6A is a prospective view illustrating an unexpanded expandable orthopedic implant device according to a sixth embodiment of the present disclosure;

FIG. 6B is a prospective view illustrating the expanded form of the expandable orthopedic implant device as depicted in FIG. 6A;

FIGS. 7A to 7E are top views illustrating different types of expandable orthopedic implant devices according to various embodiments of the present disclosure;

FIG. 8 is a prospective view illustrating partial enlarged detail of an unexpanded expandable orthopedic implant device according to an eighth embodiment of the present disclosure;

FIG. 9 is a schematic diagram illustrating the results of implanting a hip screw that applies the expandable orthopedic implant device as depicted in FIGS. 6A and 6B into a patient's femur.

### DETAILED DESCRIPTION

According to the present disclosure, an expandable orthopedic implant device is provided to increase the contact area and friction between the expandable orthopedic implant device and the peripheral tissues of the implanted bone, whereby the mechanical stability of the expandable orthopedic implant device can be improved, and the risk of superior cutout can be significantly reduced. Several embodiments of the present disclosure are disclosed below with reference to accompanying drawings.

However, the structure and content disclosed in the embodiments are for exemplary and explanatory purposes only, and the scope of protection of the present disclosure is not limited to the embodiments. Designations common to the accompanying drawings and embodiments are used to indicate identical or similar elements. It should be noted that the present disclosure does not illustrate all possible embodiments, and anyone skilled in the technology field of the invention will be able to make suitable modifications or changes based on the specification disclosed below to meet actual needs without breaching the spirit of the invention. The present disclosure is applicable to other implementations not disclosed in the specification. In addition, the drawings are simplified such that the content of the embodiments can be clearly described, and the shapes, sizes and scales of elements are schematically shown in the drawings for explanatory and exemplary purposes only, not for limiting the scope of protection of the present disclosure.

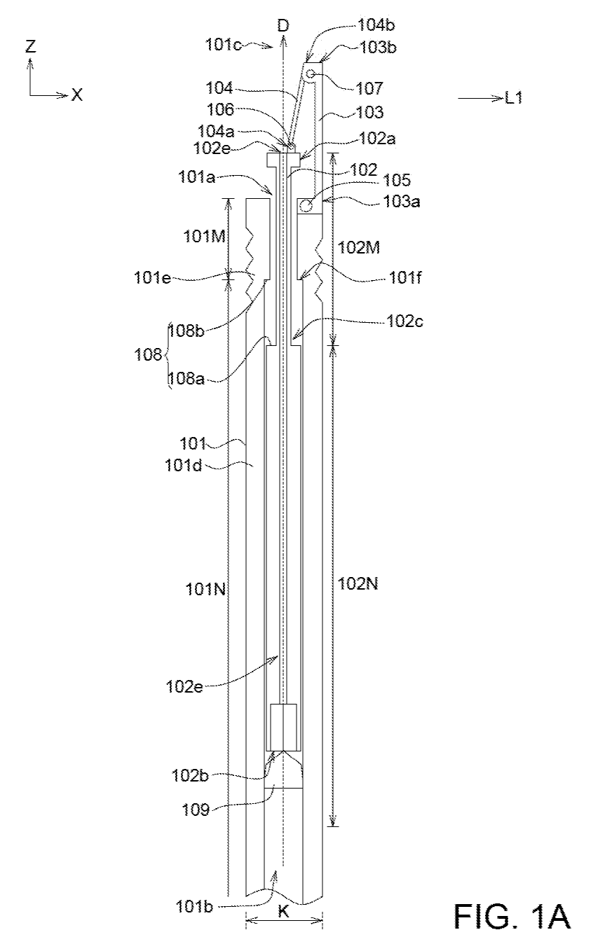

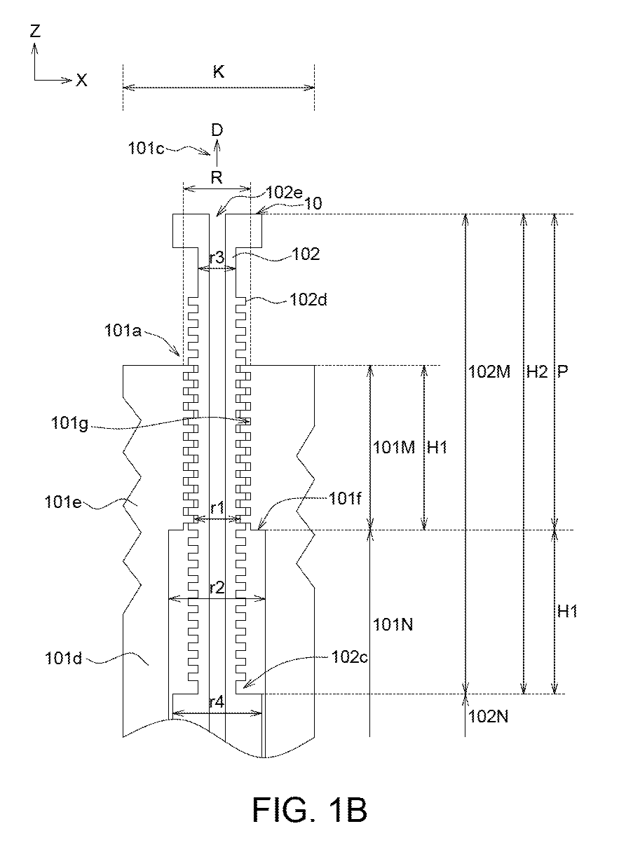

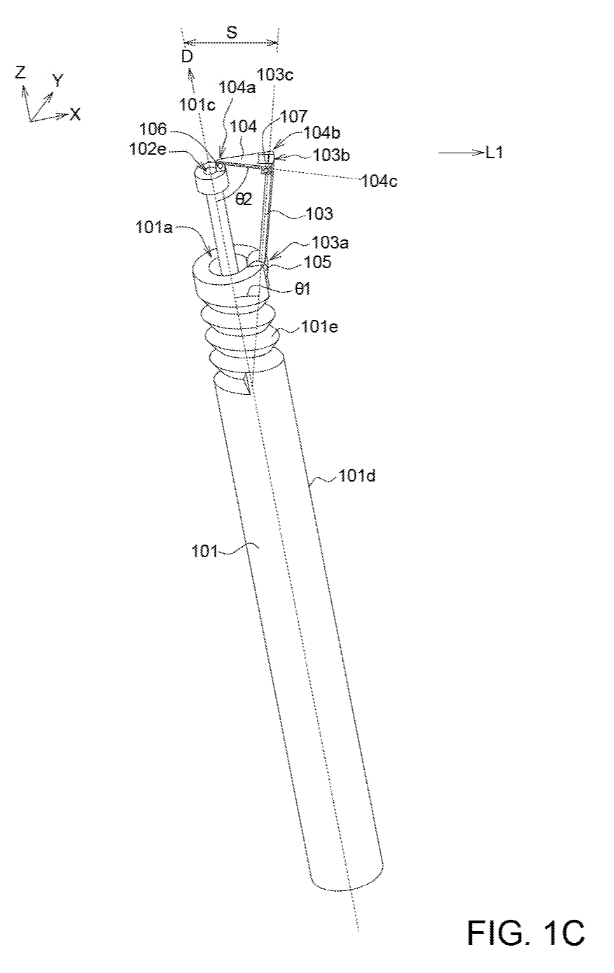

FIG. 1A is a cross-sectional view illustrating an unexpanded expandable orthopedic implant device 100 according to one embodiment of the present disclosure. FIG. 1B is a cross-sectional view illustrating partial enlarged detail of the unexpanded expandable orthopedic implant device 100 as depicted in FIG. 1A (For clearer describing purpose, the first expandable element 103 and is the first link-lever 104 originally shown in FIG. 1A are omitted). FIG. 1C is a prospective view illustrating the expanded form of the expandable orthopedic implant device 100 as depicted in FIG. 1A. The expandable orthopedic implant device 100 at least includes a casing pipe 101, a shaft 102, a first expandable element 103 and a first link-lever 104.

The casing pipe 101 can be a tubular shell having a first opening 101a, a second opening 101b and an axis 101c passing through the first opening 101a and the second opening 101b, wherein the first opening 101a and the second opening 101b are arranged at two opposite ends of the tubular shell and interconnected with each other. In some embodiments of the present disclosure, the casing pipe 101 has a cross-sectional pattern formed on the X-Y plane with various shapes, such as triangle, square, hexagon, round, oval, regular arc, irregular shape etc. In the present embodiment, the casing pipe 101 has a round-shaped cross-sectional pattern formed on the X-Y plane; the first opening 101a and the second opening 101b respectively defined by the inner edge of the sidewall 101d of the casing pipe 101 are round-shaped; and the axis 101c passing through the center of these two round-shaped openings.

The internal diameter of the casing pipe 101 may include at least two dimensions. For example, in the present embodiment, the casing pipe 101 may be divided into two portions, one is a first portion 101M, and the other is a second portion 101N connecting to the first portion 101M at a first connection location 101f. The first portion 101M that includes the portion of the casing pipe 101 extending from the first opening 101a for a distance H1 to the first connection location 101f has an internal diameter r1 substantially equal to the internal diameter R of the first opening 101a. The second portion 101N that includes the portion of the casing pipe 101 extending from the first connection location 101f to the second opening 101b has an internal diameter r2 substantially greater than the internal diameter R of the first opening 101a.

In some embodiments of the present disclosure, the casing pipe 101 may further include a spiral protrusion portion 101e protruding from the outer edge of the sidewall 101d of the casing pipe 101 and surrounding the axis 101c, used to increase the friction between the casing pipe 101 and the peripheral tissues of the implanted bone. The first portion 101M of the casing pipe 101 (the portion of the casing pipe 101 extending from the first opening 101a to the first connection location 101f) may further include a spiral guide groove 101g concaved on the inner edge of the sidewall 101d.

The shaft 102 has a head portion 102a disposed on one end of the shaft 102 and a tail portion 102b opposite to the head portion 102a. In some embodiments of the present disclosure, the shaft 102 may be a bar-shaped solid pole. The casing pipe 101 sheathes the shaft 102 to make the tail portion 102b of the shaft 102 passing through the first opening 101a of the casing pipe 101 and remaining the head portion 102a of the shaft 102 out of the first opening 101a. In the present embodiment, the shaft 102 can be a cylindrical pole; and the head portion 102a can be a cylindrical hub having a diameter substantially equal to that of the cylindrical pole (the shaft 102).

The diameter of the shaft 102 may vary depending upon the portions of which the casing pipe 101 sheathes. For example, in the present embodiment, the shaft 102 can be divided into two portions, one is a third portion 102M and the other is a fourth portion 102N connecting to the third portion 102M at a second connection location 102c. The third portion 102M that includes the portion of the shaft 102 extending from the head portion 102a for a second distance H2 to the second connection location 102c has a diameter r3 substantially less than the internal diameter r1 of the first portion 101M. The fourth portion 102N that includes the portion of the shaft 102 extending from the second connection location 102c to the tail portion 102b has a diameter r4 substantially greater than the internal diameter r1 of the first portion 101M and substantially less than the internal diameter r2 of the second portion 101N. The second distance H2 is substantially greater than the first distance H1.

In some embodiments of the present disclosure, the third portion 102M of the shaft 102 may further include a spiral protrusion portion 102d protruding from the outer edge of the shaft 102 which can be slidably engaged with the spiral guide groove 101g concaved on the inner edge of the sidewall 101d of the first portion 101M. When the spiral protrusion portion 102d of the shaft 102 rotates forward relating to the spiral guide groove 101g of the casing pipe 101, the head portion 102a of the shaft 102 can be driven to moving away from the first opening 101a of the casing pipe 101 along an extending direction D of the axis 101c. When the spiral protrusion portion 102d of the shaft 102 rotates reversely relating to the spiral guide groove 101g of the casing pipe 101, the head portion 102a of the shaft 102 can be driven to getting close to the first opening 101a of the casing pipe 101 along the extending direction D.

In some embodiments of the present disclosure, the expandable orthopedic implant device 100 may further include a rotation resistant design 108 disposed the fourth portion 102N of the shaft 102 and the second portion 101N of the casing pipe 101, which is a wedge structure applying the internal diameter difference of the casing pipe 101. For example, in the present embodiment, the shaft has a flange 108a protruding from the second connection location 102d; the casing pipe 101 has a wedge 108b protruding from the first connection location 101f; and the maximum protruding distance P of which the head portion 102a moving away from the first opening 101a can be predetermined by the engagement of the flange 108a and the wedge 108b. In other words, the maximum protruding distance P of the head portion 102a can be the difference of the first distance H1 and the second distance H2 (P=H2−H1).

When the head portion 102a of the shaft 102 is driven to moving away from the first opening 101a for the maximum protruding distance P, the flange 108a protruding from the second connection location 102c of the shaft 102 and the wedge 108b protruding from the first connection location 101f of the casing pipe 101 are wedged together to stop the shaft 102 rotating forward and driving the head portion 102a further moving away from the first opening 101a. There is a minus tolerance (draft angle) substantially ranging from 0.1° to 2° measured up between the second portion 101N of the casing pipe 101 and the fourth portion 102N of the shaft 102.

In some embodiments of the present disclosure, the expandable orthopedic implant device 100 may further include a bottom rotation resistant design adjacent to the tail portion 102b of the shaft 102. In the present embodiment, the bottom rotation resistant design can be a conical plug 109 inserted in the second portion 101N of the casing pipe 101 and contacting to the tail portion 102b of the shaft 102. Minus tolerance design can be applied to make the side walls of the conical plug 109 and the second portion 101N of the casing pipe 101 pressed tightly in contact with each other, so as to further prevent the shaft 102 from rotating reversely and moving backwards.

In some embodiments of the present disclosure, the shaft 102 may have a hollow tubular structure. For example, in the present embodiment, the shaft 102 can have a long through-hole 102e passing through the head portion 102a and the tail portion 102b. When the expandable orthopedic implant device 100 is implanted into a patient's bone, such as a femoral head, injectable biocompatible fillings, such as bone cement, can be injected into the femoral head through the long through-hole 102e to stimulate new bone formation.

The first expandable element 103 may include a first terminal 103a and a second terminal 103b. The first terminal 103a is pivoted on the casing pipe 101 through a first pivot bolt 105; and the second terminal 103b is pivoted on the first link-lever 104. In some embodiments of the present embodiments, the first expandable element 103 may have a plate structure, a rod-shaped structure, vertebral structure, or the arbitrary combinations thereof. For example, in the present embodiment, the first expandable element 103 can be a rod-shaped structure having a first lever arm 103c perpendicular to the first pivot bolt 105 and forming an angle Θ1 with the axis 101c of the casing pipe 101, wherein the angle Θ1 can vary depending upon the related rotating position of the first expandable element 103 against the first pivot bolt 105. In the present embodiment, the angle Θ1 can range from 0° to 70°.

The first link-lever 104 has a first proximal end 104a and a first distal end 104b, wherein the first proximal end 104a is pivoted to the head portion 102a of the shaft 102 through a second pivot bolt 106. In the present embodiment, the first link-lever 104 has a second lever arm 104c perpendicular to the second pivot bolt 106; and the first distal end 104b of the first link-lever 104 and the second terminal 103b of the first expandable element 103 are pivoted with each other at an intersectional point 107 of the first lever arm 103c and the second lever arm 104c. The shaft 102 and the second lever arm 104c of the first link-lever 104 can from an angle Θ2 varying depending upon the related rotating position of the first link-lever 104 against the second pivot bolt 106. In the present embodiment, the angle Θ2 can range from 175° to 90°.

When the shaft 102 rotates forward, the head portion 102a of the shaft 102 can be driven to moving away from the first opening 101a along the extending direction D of the axis 101c, and the first link-lever 104 can be pushed by the shaft 102 to enact the second terminal 103b of the first expandable element 103 moving away from the axis 101c of the casing pipe 101 along a first direction L1 perpendicular to the axis 101c. Such that, the second terminal 103b of the first expandable element 103 and the first distal end 104b of the first link-lever 104 can radially expand outward from the axis 101c. In contrast, when the shaft 102 rotates reversely, the head portion 102a of the shaft 102 can be driven to getting close to the first opening 101a along the extending direction D of the axis 101c, and the first link-lever 104 can be drawn by the shaft 102 to pull the second terminal 103b of the first expandable element 103 getting close to the axis 101c of the casing pipe 101 along a first direction L1 perpendicular to the axis 101c. Such that, the second terminal 103b of the first expandable element 103 and the first distal end 104b of the first link-lever 104 can radially retracted inward to the axis 101c.

In some embodiments of the present disclosure, when the first expandable element 103 and the first link-lever 104 are retracted to a position most adjacent to the axis 101c, the first expandable element 103 and the first link-lever 104 can be round up in the region defined by the outer diameter K of the casing pipe 101. When the first expandable element 103 and the first link-lever 104 expand to the maximum, the second terminal 103b of the first expandable element 103 (which is pivoted with the first distal end 104b of the first link-lever 104) can be moved out of the region defined by the outer diameter K of the casing pipe 101. In some embodiments, the ratio of the distance S between the first expandable element 103 (which is pivoted with the first distal end 104b of the first link-lever 104) and the axis 101c of the casing pipe 101 to the outer diameter K of the casing pipe 101 (i.e. S/K) may substantially range from 0.1 to 3.5.

For example, in the present embodiment, when the first expandable element 103 and the first link-lever 104 are retracted to a position most adjacent to the axis 101c, the distance S between the first expandable element 103 and the axis 101c can be 0.6 time of the outer diameter K of the casing pipe 101. When the first expandable element 103 and the first link-lever 104 expand to the maximum, the distance S between the first expandable element 103 and the axis 101c can be 3 time of the outer diameter K of the casing pipe 101. In some other embodiments, the extent of expansion of the first expandable element 103 can be adjusted depending upon the lengths of the first expandable element 103 and the first link-lever 104 as well as the angles that are formed by one of the first expandable element 103 and the first link-lever 104 and one of the casing pipe 101 and the shaft 102.

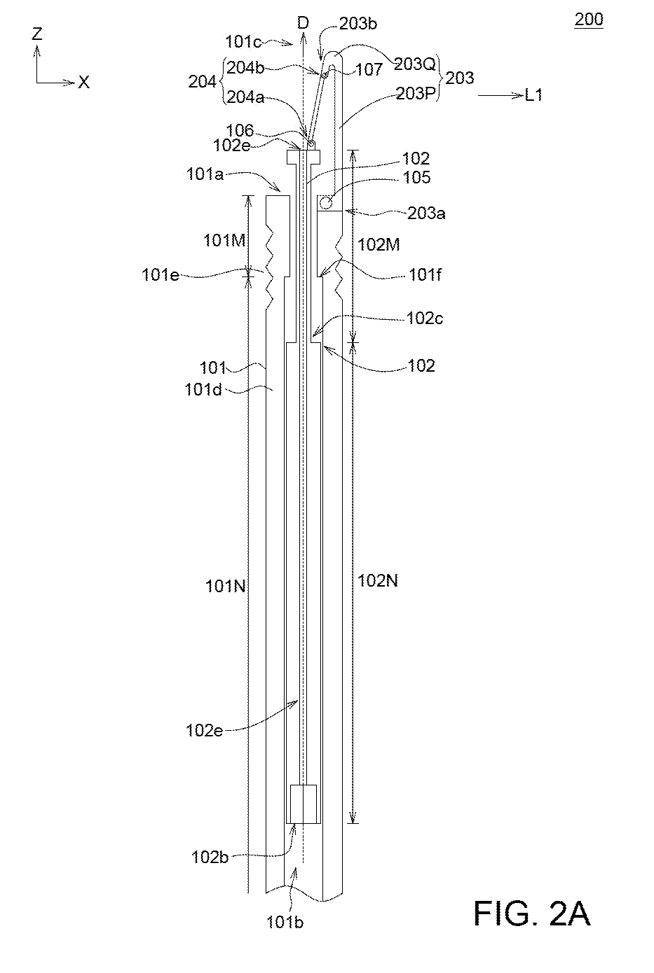

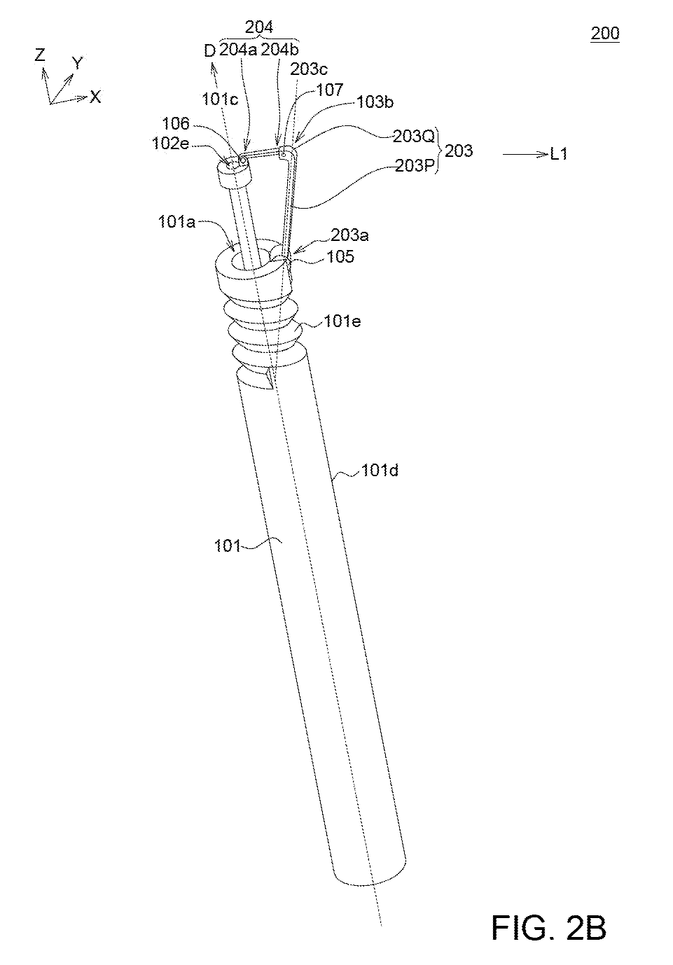

However, the connection of the first expandable element 103 and the first link-lever 104 is not limited to this regard. FIG. 2A is a cross-sectional view illustrating an unexpanded expandable orthopedic implant device 200 according to another embodiment of the present disclosure; and FIG. 2B is a prospective view illustrating the expanded form of the expandable orthopedic implant device 200 as depicted in FIG. 2A. The structure of the expandable orthopedic implant device 200 is like that of the expandable orthopedic implant device 100 as depicted in FIGS. 1A to 1C except that the first expandable element 203 of the expandable orthopedic implant device 200 has different design.

In the present embodiment, the first expandable element 203 has an element body 203P and a first curved part 203Q connecting with each other. The element body 203P has a first lever arm 203c perpendicular to the first pivot bolt 105 and a first terminal 203a pivoted to the casing pipe by the first pivot bolt 105. The first curved part 203Q has a second terminal departing away from the first lever arm 203c. In comparison with the expandable orthopedic implant device 100, when the first expandable element 203 and the first link-lever 204 are retracted to a position most adjacent to the axis 101c of the casing pipe 101, the second terminal 203b of the first expandable element 203 and the first distal end 204b of the first link-lever 204 may getting closer to the axis 101c of the casing pipe 101 than the second terminal 103b and the first distal end 104b of the expandable orthopedic implant device 100 do. The expandable orthopedic implant device 200 can have a smaller radial cross-sectional area than that of the expandable orthopedic implant device 100, when they are in its unexpanded form. Such that, the expandable orthopedic implant device 200 can be implanted into the bone easier than the expandable orthopedic implant device 100.

Of note, nevertheless the first curved part 203Q of the first expandable element 203 depicted in FIGS. 2A and 2B is shaped as an arc; the appearance of the first curved part 203Q may not be limited to this regard. Any bent-rod structure or curved structure that can make the second terminal 203b of the first expandable element 203 departing from the first lever arm 203c may not deviate from the spirit of the of the present invention.

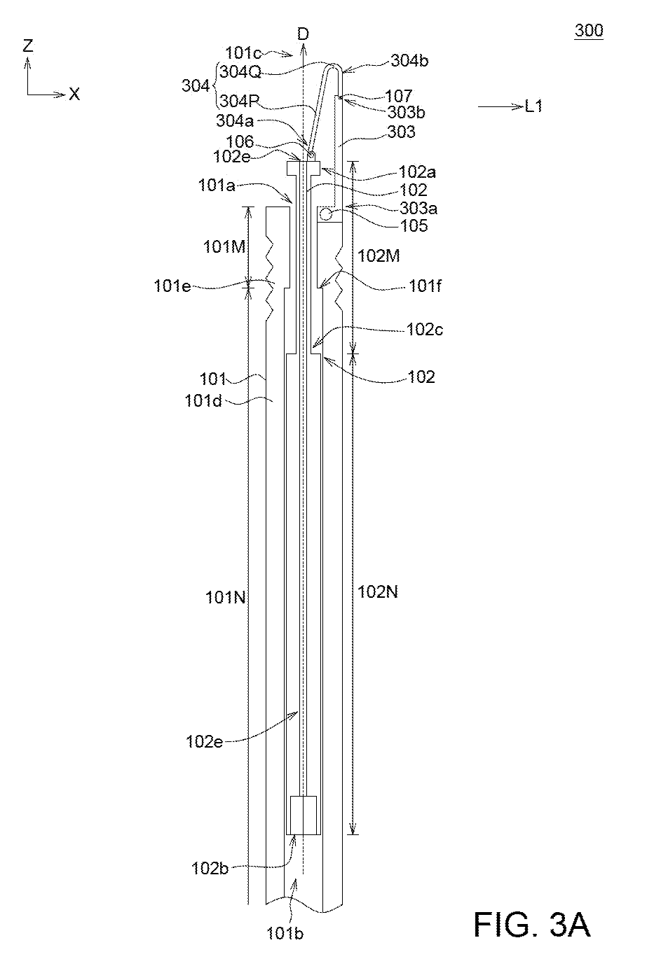

FIG. 3A is a cross-sectional view illustrating an unexpanded expandable orthopedic implant device 300 according to yet another embodiment of the present disclosure; FIG. 3B is a prospective view illustrating the expanded form of the expandable orthopedic implant device 300 as depicted in FIG. 3A. The structure of the expandable orthopedic implant device 300 is like that of the expandable orthopedic implant device 100 as depicted in FIGS. 1A to 1C except that the first link-lever 304 of the expandable orthopedic implant device 300 has different design.

In the present embodiment, the first link-lever 304 has a lever body 304P and a second curved part 304Q connecting with each other. The second curved par 304Q has a second lever arm 304c perpendicular to the second pivot bolt 106 and a first proximal end 104a pivoted with the head portion 102a of the shaft 102 by the second pivot bolt 106. The second curved part 304Q has a first distal end 304b departing away from the second lever arm 304c and pivoted to the second terminal 303b of the first expandable element 303. In comparison with the expandable orthopedic implant device 100, when the first expandable element 303 and the first link-lever 304 are expanded to the maximum, the second terminal 303b of the first expandable element 303 and the first distal end 304b of the first link-lever 304 may depart from the axis 101c of the casing pipe 101 far more than the second terminal 103b and the first distal end 104b of the expandable orthopedic implant device 100 do. The expandable orthopedic implant device 300 can have greater radial cross-sectional area than that of the expandable orthopedic implant device 100, as the head portion 102a of the shaft 102 departing away the first opening of the casing pipe 101 for the same distance.

Of note, nevertheless the second curved part 304Q of the first link-lever 304 depicted in FIGS. 3A and 3B is shaped as an arc; the appearance of the second curved part 304Q may not be limited to this regard. Any bent-rod structure or curved structure that can make the first distal end 304b of the first link-lever 304 departing from the second lever arm 304c may not deviate from the spirit of the of the present invention.

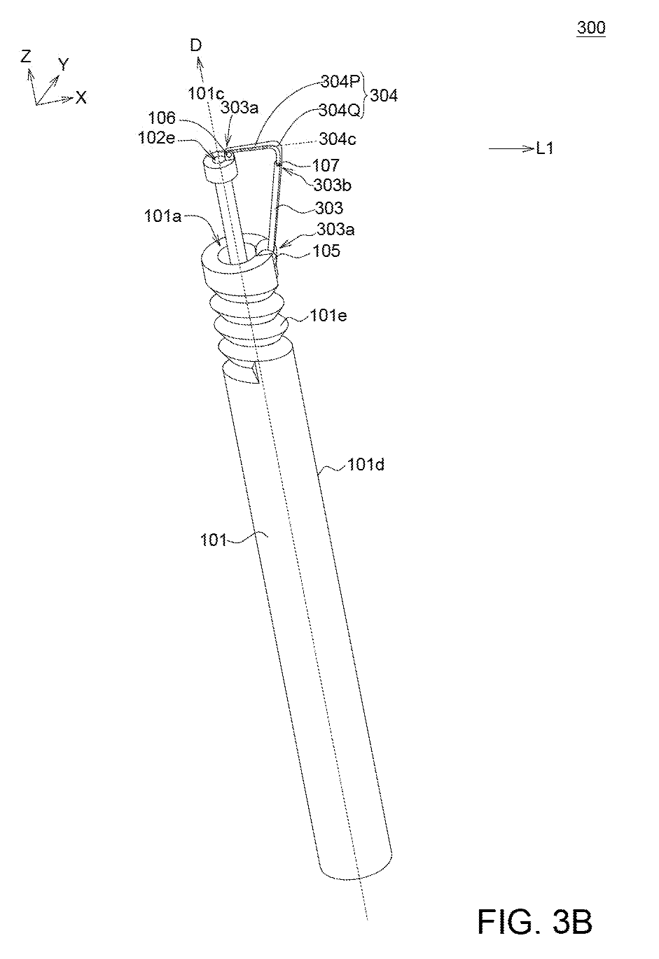

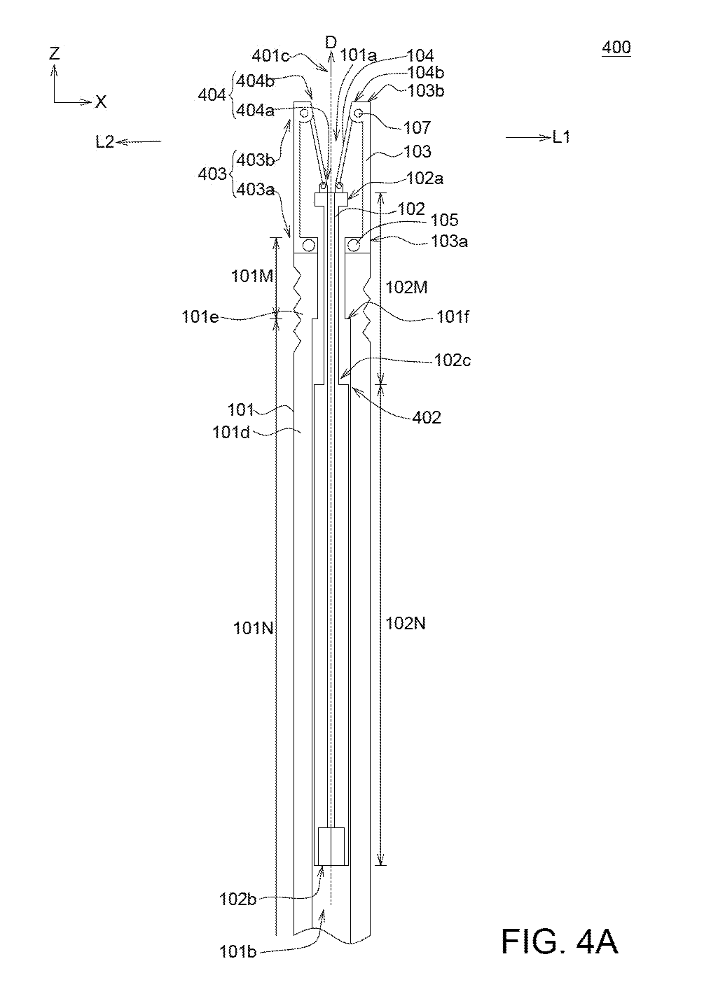

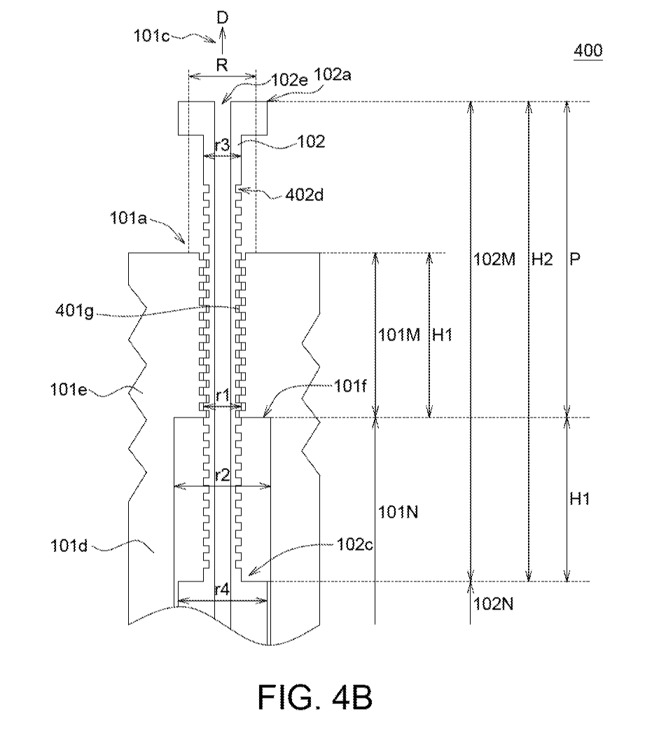

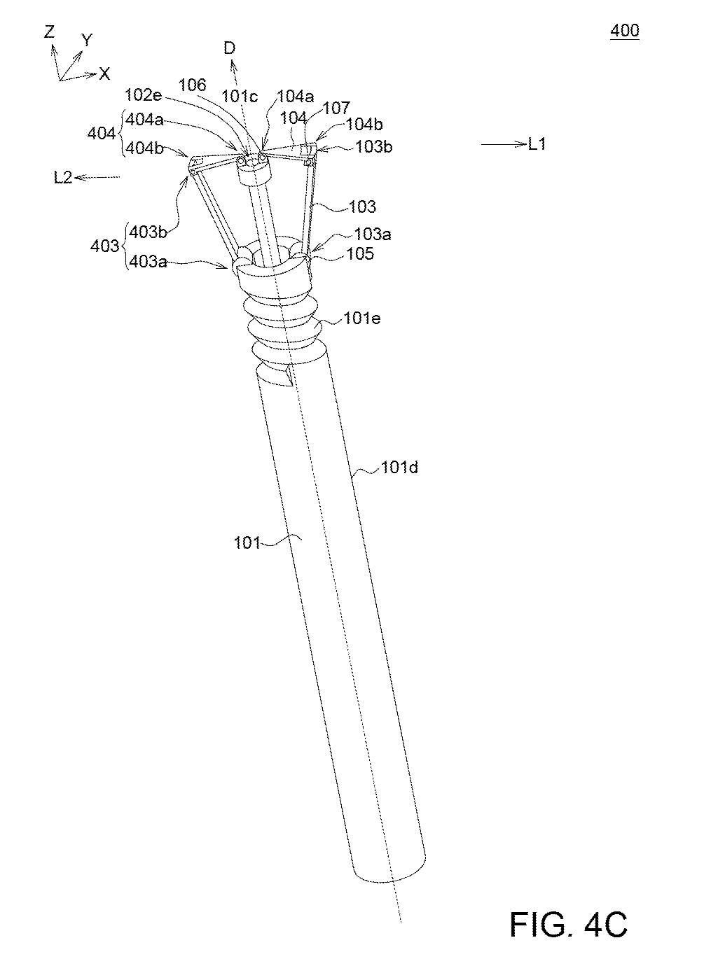

FIG. 4A is a cross-sectional view illustrating an unexpanded expandable orthopedic implant device 400 according to a fourth embodiment of the present disclosure. FIG. 4B is a cross-sectional view illustrating partial enlarged detail of the unexpanded expandable orthopedic implant device 400 as depicted in FIG. 4A (For clearer describing purpose, the second expandable element 403 and is the second link-lever 404 originally shown in FIG. 4A are omitted). FIG. 4C is a prospective view illustrating the expanded form of the expandable orthopedic implant device 400 as depicted in FIG. 4A.

The structure of the expandable orthopedic implant device 400 is like that of the expandable orthopedic implant device 100 as depicted in FIGS. 1A to 1C except that the expandable orthopedic implant device 400 further includes a second expandable element 403 and a second link-lever 404. The second expandable element 403 includes a third terminal 403a and a fourth terminal 403b, wherein the third terminal 403a is pivoted to the casing pipe 101. The second link-lever 404 has a second proximal end 404a pivoted to the head portion 102a of the shaft 102 and a second distal end 404b pivoted to the fourth terminal 403b.

In addition, the expandable orthopedic implant device 400 may further include a spiral guide groove 402d concaved on the third portion 102M of the shaft 102 and slidably engaged with a spiral protrusion portion 401g protruding from the inner edge of the first portion 101M of the casing pipe 101. When the shaft 102 rotates forward to drive the head portion 102a of the shaft 102 moving away from the first opening 101a of the casing pipe 101 along the extending direction D of the axis 101c, the second link-lever 404 can be pushed by the shaft 102 to enact the fourth terminal 403b of the second expandable element 403 moving away from the axis 101c of the casing pipe 101 along a second direction L2 perpendicular to the axis 101c.

In the present embodiment, the first direction L1 can be parallel and opposite to the second direction L2. In other words, the first direction L1 and the second direction L2 form an angle substantially equal to 180°. However, in some other embodiments, the expanding direction of the second expandable element 403 and the second link-lever 404 may be adjusted depends upon the bone density and the stress applied to the bone in which the expandable orthopedic implant device 500 is implanted, to make the first direction L1 and the second direction L2 form a non-straight angle Θ5, substantially less than to 180° (see FIG. 5).

Although, the dimension (length/width) of the first expandable element 103 is identical to that of the second expandable element 403; and the dimension of the first link-lever 104 is identical to that of the second link-lever 404, as depicted in FIGS. 4A to 4C and 5, but the dimensions of the first expandable element 103, the second expandable element 403, the first link-lever 104 and the second link-lever 404 are not limited to these regards. In some other embodiments, the dimensions of the first expandable element 103, the second expandable element 403, the first link-lever 104 and the second link-lever 404 may be varied depends upon the bone density and the stress applied to the bone in which the expandable orthopedic implant device 500 is implanted. For example, the dimension of the first expandable element 103 may be different from that of the second expandable element 403; and the dimension of the first link-lever 104 may be different from that of the second link-lever 404 (not shown).

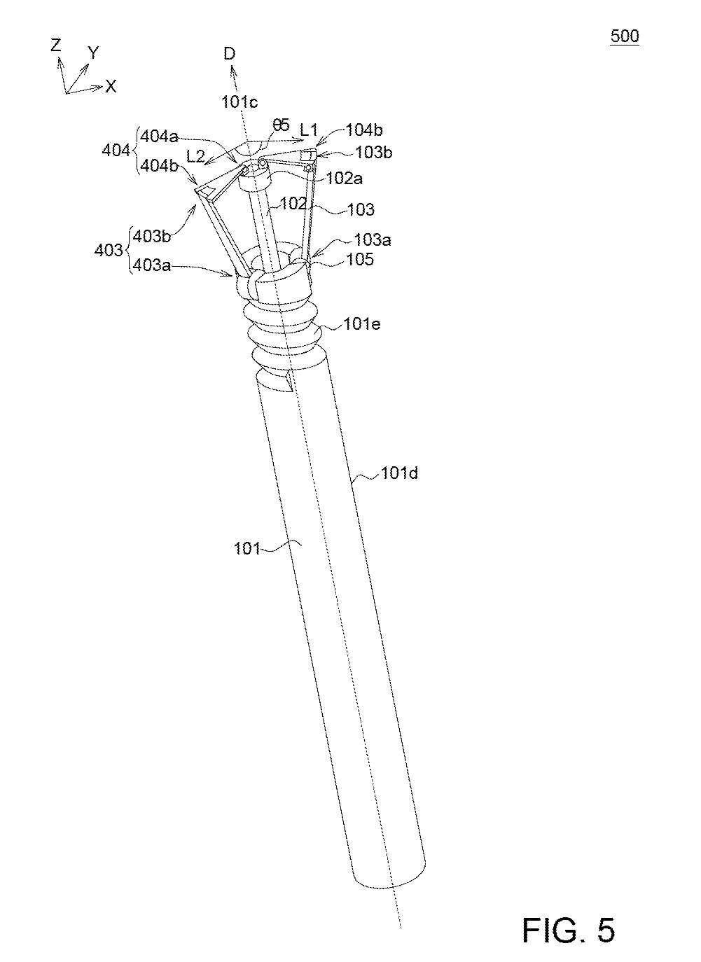

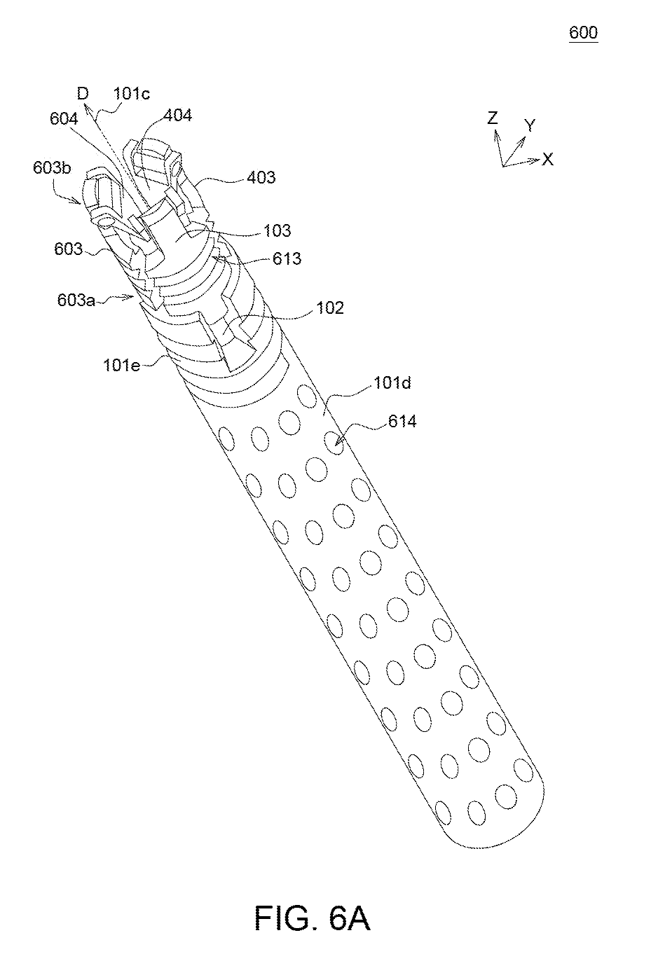

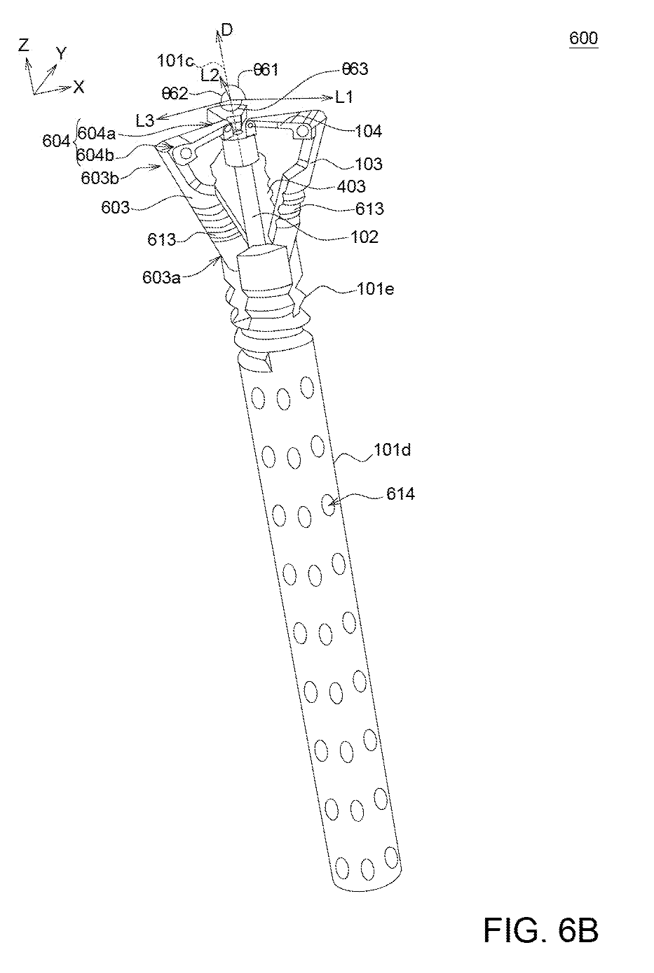

FIG. 6A is a prospective view illustrating an unexpanded expandable orthopedic implant device 600 according to a sixth embodiment of the present disclosure. FIG. 6B is a prospective view illustrating the expanded form of the expandable orthopedic implant device 600 as depicted in FIG. 6A. The structure of the expandable orthopedic implant device 600 is like that of the expandable orthopedic implant device 400 as depicted in FIG. 4C except that the expandable orthopedic implant device 600 further includes a third expandable element 603 and a third link-lever 604.

The third expandable element 603 includes a fifth terminal 603a and a sixth terminal 603b, wherein the fifth terminal 603a is pivoted to the casing pipe 101. The third link-lever 604 has a third proximal end 604a pivoted to the head portion 102a of the shaft 102 and a third distal end 604b pivoted to the sixth terminal 603b. When the shaft 102 rotates forward to drive the head portion 102a of the shaft 102 moving away from the first opening 101a of the casing pipe 101 along the extending direction D of the axis 101c, the third link-lever 604 can be pushed by the shaft 102 to enact the six terminal 603b of the third expandable element 603 moving away from the axis 101c of the casing pipe 101 along a third direction L3 perpendicular to the axis 101c.

In the present embodiment, the first direction L1 and the second direction L2 form an angle Θ61; the second direction L2 and the third direction L3 form an angle Θ62; the first direction L1 and the third direction L3 form an angle Θ63; and the angles Θ61, Θ62 and Θ63 form a round angle (360°), wherein the angles Θ61, Θ62 and Θ63 are equal to each other (i.e. each of which is 120°). Similarly, the expanding direction of the third expandable element 603 and the third link-lever 604 may be adjusted depends upon the bone density and the stress applied to the bone in which the expandable orthopedic implant device 600 is implanted, to make the angles Θ61, Θ62 and Θ63 has different magnitudes. For example, in some other embodiments, at least two the angles Θ61, Θ62 and Θ63 may be different form each other. In one embodiment, two of the angles Θ61, Θ62 and Θ63 have the same magnitude which is different from that of the other one.

To improve the stress resistance of the expandable orthopedic implant device 600, in some embodiments of the present disclosure, each of the first expandable element 103, the second expandable element 403 and the third expandable element 603 may be configured as an arc-shaped plate structure. The first expandable element 103, the second expandable element 403 and the third expandable element 603 can be parallel to the sidewall 101d of the casing pipe 101, when they are radially retracted inward to the axis 101c. Each of the first expandable element 103, the second expandable element 403 and the third expandable element 603 can also includes a spiral protrusion portion 613 protruding from the outer edge thereof departing away from the axis 101c, wherein the spiral protrusion portion 613 and the spiral protrusion portion 101e that protrudes from the outer edge of the sidewall 101d of the casing pipe 101 has the same axis of symmetry.

In some embodiments of the present disclosure, the expandable orthopedic implant device 600 may be made of biodegradable material, such as a biodegradable component consisting of an iron (Fe)-base material, a magnesium (Mg)-base material, a manganese (Mn)-containing material or the arbitrary combinations thereof. To increase the biodegradability of the expandable orthopedic implant device 600, at least one through-hole 614 may be formed in at least one of the casing pipe 101, the shaft 102, the first expandable element 103, the second expandable element 403 and the third expandable element 603. In the present embodiment, the casing pipe 101 has a plurality of through-holes 614 passing therethrough to enhance the local biodegradability.

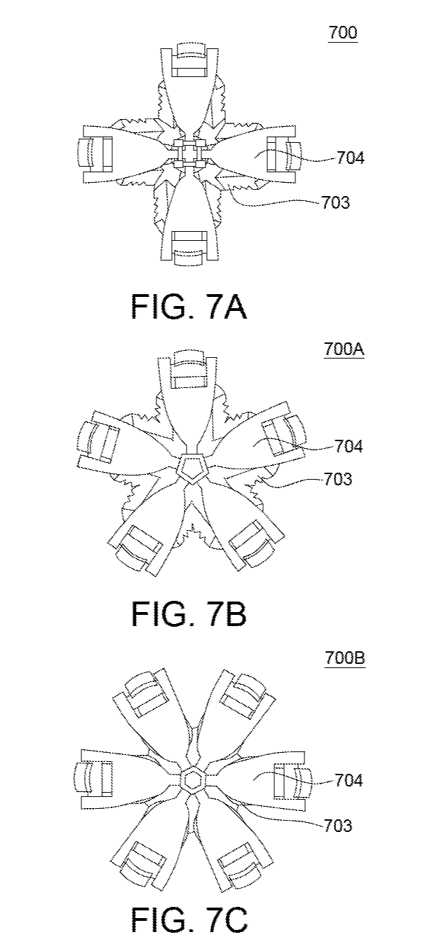

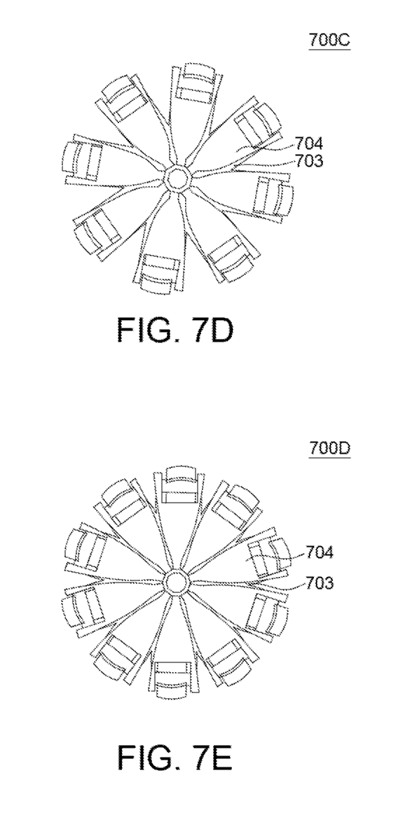

In should be appreciated that the number of the expandable element and the link-lever involved in the expandable orthopedic implant device 600 may not limited to this regard. Within the maximum accommodating space, the expandable orthopedic implant device 600 may include more expandable elements and link-levers. For example, FIGS. 7A to 7E are top views illustrating different types of expandable orthopedic implant devices 700, 700A, 700B, 700C and 700D according to various embodiments of the present disclosure. The expandable orthopedic implant devices 700, 700A, 700B, 700C and 700D respectively have four sets, five sets, six sets, seven sets and eight sets of anchor structure, each of which includes one expandable element 703 and one link-lever 704.

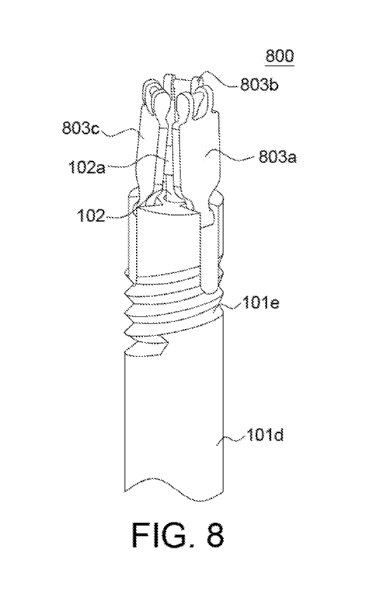

The appearance of the expandable element and the link-lever involved in the expandable orthopedic implant device 600 may not limited to this regard. FIG. 8 is a prospective view illustrating partial enlarged detail of an unexpanded expandable orthopedic implant device 800 according to an eighth embodiment of the present disclosure. The structure of the expandable orthopedic implant device 800 is like that of the expandable orthopedic implant device 600 as depicted in FIGS. 6A and 6B except that each of the first expandable element 803a, the second expandable element 803b and the third expandable element 803c involved in the expandable orthopedic implant device 800 may be a flat-plate structure.

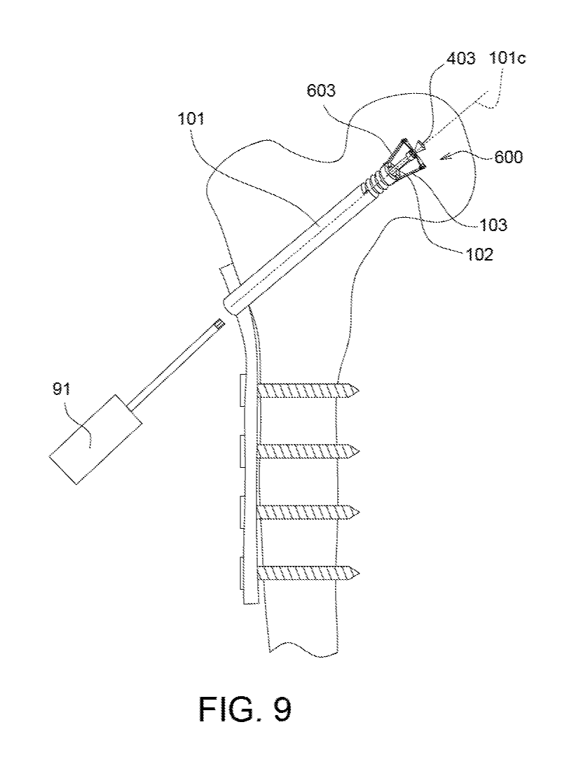

FIG. 9 is a schematic diagram illustrating the results of implanting a hip screw that applies the expandable orthopedic implant device 600 as depicted in FIGS. 6A and 6B into a patient's femur. After the hip screw applying the expandable orthopedic implant device 600 is implanted in the predetermined position of the patient's femur, a tool 91 can be used to drive the shaft 102 rotating forward and enact the first expandable element 103, the second expandable element 403 and the third expandable element 603 radially expanding outward from the axis 101c, whereby the contact area and friction between the expandable orthopedic implant device 600 and the peripheral tissues of the patient's femur can be increased to prevent the expandable orthopedic implant device 600 from rotating or bonding loosening. As a result, the mechanical stability of the expandable orthopedic implant device 600 implanted in the patient's femur can be improved, and the risk of superior cutout can be significantly reduced.

However, the application of the expandable orthopedic implant devices as provided by the embodiments above may not limited to this regard. The expandable orthopedic implant devices can be also applied to any orthopedic surgery to provide tissue fixing, connection, filling, supporting, stretching or other suitable mechanical and physiological functions. For example, the expandable orthopedic implant devices as provided by the embodiments above may be applied to expandable bone nails, expandable intervertebral cages, expandable pedicle screws or other suitable orthopedic implant devices.

According to another embodiment of the present disclosure, an expandable orthopedic implant device including a shaft, a casing pipe sheathing one end of the shaft, an expandable element and a link-lever is provided, wherein a distal end of the link-lever is pivoted to a second terminal of the expandable element, and a proximal end of the link-lever and a first terminal of the expandable element opposite to the second terminal are respectively pivoted to the shaft and the casing pipe. When the second terminal of the expandable element is driven by the link-lever enacted by the shaft moving away from the axis along a direction perpendicular to the axis, an anchor structure can be formed by the expandable element and the link-lever to increase the contact area and friction between the expandable orthopedic implant device and the peripheral tissues of the implanted bone, so as to prevent the expandable orthopedic implant device from rotating or bonding loosening, whereby the mechanical stability of the expandable orthopedic implant device implanted in bones can be improved, and the risk of superior cutout can be significantly reduced.

While the invention has been described by way of example and in terms of the preferred embodiment (s), it is to be understood that the invention is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. An expandable orthopedic implant, comprising:
a casing pipe, having a first opening, a second opening and an axis passing through the first opening and the second opening;
a shaft, wherein the casing pipe sheathes one end of the shaft, and the other end of the shaft has a head portion disposed out of the first opening;
a first expandable element, having a first terminal pivoted on the casing pipe and a second terminal; and
a first link-lever, having a first proximal end pivoted to the head portion and a first distal end pivoted to the second terminal;
when the head portion is driven moving away from the first opening along an extending direction of the axis, the first link-lever is pushed by the shaft to enact the second terminal moving away from the axis along a first direction perpendicular to the axis.

2. The expandable orthopedic implant according to claim 1, wherein the shaft and the first link-lever from an angle ranging from 175° to 90°.

3. The expandable orthopedic implant according to claim 1, wherein the first terminal is pivoted on the casing pipe through a first pivot bolt; and the first expandable element has a first lever arm perpendicular to the first pivot bolt and forming an angle ranging from 0° to 70° with the axis of the casing pipe.

4. The expandable orthopedic implant according to claim 3, wherein the first link-lever is pivoted to the head portion through a second pivot bolt and has a second lever arm perpendicular to the second pivot bolt; and the first distal end and the second terminal are pivoted with each other at an intersectional point of the first lever arm and the second lever arm.

5. The expandable orthopedic implant according to claim 1, wherein the first expandable element has an element body and a first curved part connecting with each other; the first terminal is disposed on the element body that has the first lever arm, and pivoted to the casing pipe by a first pivot bolt perpendicular to the first lever arm; and the second terminal is disposed on the first curved part and departs away from the first lever arm.

6. The expandable orthopedic implant according to claim 1, wherein the first link-lever has a lever body and a second curved part connecting with each other; the first proximal end is disposed on the lever body that has a second lever arm, and pivoted with the head portion by a second pivot bolt perpendicular to the second lever arm; the first distal end is disposed on the second curved part and departs away from the second lever arm.

7. The expandable orthopedic implant according to claim 1, wherein the casing pipe comprises a plurality of through-holes disposed in at least one sidewall used to define the first opening and the second opening.

8. The expandable orthopedic implant according to claim 1, wherein the first expandable element is configured as an arc-shaped plate structure parallel to at least one sidewall of the casing pipe used to define the first opening and the second opening.

9. The expandable orthopedic implant according to claim 8, wherein the first expandable element comprises a first spiral protrusion portion protruding from an outer edge of the first expandable element departing away the axis.

10. The expandable orthopedic implant according to claim 9, wherein the casing pipe comprises a second spiral protrusion portion protruding from an outer edge of the at least one sidewall and surrounding the axis.

11. The expandable orthopedic implant according to claim 1, further comprising:
a third spiral protrusion portion protruding from an inner edge of at least one sidewall of the casing pipe used to define the first opening and the second opening; and
a first spiral guide groove concaved on a surface of the shaft and slidably engaged with the third spiral protrusion.

12. The expandable orthopedic implant according to claim 1, further comprising:
a second spiral guide groove concaved on an inner edge of at least one sidewall of the casing pipe used to define the first opening and the second opening; and
a fourth spiral protrusion portion protruding from a surface of the shaft and slidably engaged with the second spiral guide groove.

13. The expandable orthopedic implant according to claim 1, wherein the shaft is a hollow tubular structure having a long through-hole passing through the head portion.

14. The expandable orthopedic implant according to claim 1, wherein
the casing pipe is divided into a first portion and a second portion connecting to the first portion; the first portion has a first internal diameter substantially equal to a diameter of the first opening; the second portion has a second internal diameter substantially greater than the diameter of the first opening; and
the shaft is divided into a third portion and a fourth portion connecting to the third portion; the third portion has a first diameter substantially less than the first internal diameter; the fourth portion has a second diameter substantially greater than the first internal diameter and less than the second internal diameter.

15. The expandable orthopedic implant according to claim 14, wherein the casing pipe has a wedge protruding from a first connection location at which the second portion connects to the first portion; and the shaft has a flange protruding from a second connection location at which the third portion connects to the fourth portion.

16. The expandable orthopedic implant according to claim 1, further comprising a bottom rotation resistant design inserted in the casing pipe and contacting to a tail portion of the shaft.

17. The expandable orthopedic implant according to claim 1, further comprising:
a second expandable element having a third terminal and a fourth terminal, wherein the third terminal is pivoted to the casing pipe; and
a second link-lever having a second proximal end pivoted to the head portion and a second distal end pivoted to the fourth terminal;
when the head portion is driven moving away from the first opening, the second link-lever is pushed by the shaft to enact the fourth terminal moving away from the axis along a second direction perpendicular to the axis.

18. The expandable orthopedic implant according to claim 17, wherein the first direction and the second direction form a straight angle or a non-straight angle.

19. The expandable orthopedic implant according to claim 17, further comprising:
a third expandable element having a fifth terminal and a sixth terminal, wherein the fifth terminal is pivoted to the casing pipe; and
a third link-lever having a third proximal end pivoted to the head portion and a third distal end pivoted to the sixth terminal;
when the head portion is driven moving away from the first opening, the third link-lever is pushed by the shaft to enact the sixth terminal moving away from the axis along a third direction perpendicular to the axis.

20. The expandable orthopedic implant according to claim 18, wherein the first direction and the second direction form a first angle; the second direction and the third direction form a second angle; and the first angle is equal to or greater than the second angle.
